(19) United States
(12) Patent Application Publication
(10) Pub. No.: US 2024/0216604 A1
(43) Pub. Date: Jul. 4, 2024

(54) PCA PUMP PROGRAMMING AND PATIENT HISTORY USER INTERFACE

(71) Applicants: Baxter International Inc., Deerfield, IL (US); Baxter Healthcare SA, Glattpark (Opfikon) (CH)

(72) Inventor: Rajesh Swarnkar, Bangalore (IN)

(21) Appl. No.: 18/396,281

(22) Filed: Dec. 26, 2023

(30) Foreign Application Priority Data
Dec. 28, 2022 (IN) 202241076492

(51) Int. Cl. A61M 5/142

(52) U.S. Cl. CPC A61M 5/142; A61M 2005/1405

(57) ABSTRACT

Example systems, methods, and apparatus are disclosed herein for on-screen parameter programming guidance based on user-inputted data and patient history graphical display generation based on a user-inputted command. The example systems, methods, and apparatus are configured to use a preloaded drug library to determine the upper and lower limits of parameters, and display a color graph and differently-colored text on a PCA pump user interface screen accordingly. Additionally, the example systems, methods, and apparatus are configured to use real-time patient history data to generate and display a patient history graph on a user interface screen. The disclosed systems, methods, and apparatus prevent human programming errors by minimizing the need for extensive user interaction with PCA pumps and the need to toggle between programming screens. Further, the disclosed systems, methods, and apparatus prevent human programming errors by displaying patient history in a single, consolidated graph.

### PRIORITY CLAIM AND CROSS-REFERENCE TO RELATED APPLICATIONS

The present application claims priority to Indian Patent Application No. 202241076492, filed on Dec. 28, 2022, and entitled PCA PUMP PROGRAMMING AND PATIENT HISTORY USER INTERFACE, the contents of which are hereby incorporated by reference in their entirety.

### BACKGROUND

Patient Control Analgesia (PCA) pumps are commonly used for administering pain medication intravenously. PCA pumps are programmed to deliver an infusion at a continuous constant rate or can include Patient Control Analgesia (PCA) boluses. To administer an infusion, a user programs the PCA pump and understands the therapy history of the patient. PCA pumps include multiple parameters to be programmed by the user, and each parameter has certain acceptable ranges (upper lower and upper limits, with soft and hard limits). The parameters are interrelated, which makes programming more challenging and leads to programming errors that can delay therapy administration and lead to patient discomfort. Currently, PCA pumps display a user interface for programming each parameter individually. However, the user has to toggle between screens to program infusion therapies since the single screen does not display the other parameters or the interrelation with the other parameters. As such, the user spends considerable time toggling between screens until the user can correctly program the parameters.

Relatedly, once an infusion therapy begins, the user must be able to access the patient therapy history (patient history), which is logged in the PCA pump. The patient history includes six or seven parameters that are viewed by the user to make the best clinical decision at the patient's bedside. Currently, PCA pump interfaces display the patient history in multiple screens and in a non-graphical format. Additionally, current PCA pump interfaces only show patient history for a specific hour, but the user often needs a patient history for 1 hr, 2 hr, 4 hr, 8 hr, 12 hr, 24 hr, etc. As such, current PCA pump interfaces do not have the ability to show all patient history in one place, which makes programming more challenging and can lead to programming errors that can delay therapy administration and result in patient discomfort.

A need accordingly exists for a PCA pump interface that provides on-screen programming guidance for multiple interrelated parameters and displays patient history in a graphical format on a single screen.

### SUMMARY

Example systems, methods, and apparatus are disclosed herein for on-screen parameter programming guidance based on user-inputted data and patient history graphical display generation based on a user-inputted command. The example systems, methods, and apparatus are configured to use a preloaded drug library to determine upper and lower limits of parameters, and display a color graph and differently-colored text on a PCA pump user interface screen accordingly. Additionally, the example systems, methods, and apparatus are configured to use real-time patient history data to generate and display a patient history graph on a user interface screen. The disclosed systems, methods, and apparatus prevent human programming errors by minimizing the need for extensive user interaction with PCA pumps and the need to toggle between programming screens. Further, the disclosed systems, methods, and apparatus prevent human programming errors by displaying patient history in a single, consolidated graph. As such, the disclosed systems, methods, and apparatus increase efficient infusion administration and prevent programming errors that might result in patient discomfort or injury.

In light of the disclosure herein, and without limiting the scope of the invention in any way, in a first aspect of the present disclosure, which may be combined with any other aspect listed herein unless specified otherwise, a PCA pump includes an actuator, a syringe holder, a display screen, a memory, and a processor. The memory stores instructions that when executed by the processor, cause the processor to: receive a user-selected infusion therapy type and a user-inputted parameter value, determine the lower and upper limits of the parameter type associated with the user-inputted parameter value and user-selected infusion therapy type, compare the user-inputted parameter value to the lower and upper limits of the parameter type, generate a graph on the display screen that shows the lower and upper limits of the parameter type in different colors, and generate different-colored text for the user-inputted parameter value based on where the user-inputted parameter value falls within the lower and upper limits of the parameter type.

In a second aspect of the present disclosure, which may be combined with any other aspect listed herein unless specified otherwise, the upper and lower limits of the parameter type include the soft limits and hard limits.

In a third aspect of the present disclosure, which may be combined with any other aspect listed herein unless specified otherwise, the graph is a bar graph.

In a fourth aspect of the present disclosure, which may be combined with any other aspect listed herein unless specified otherwise, the different colors in the bar graph correspond with the lower and upper limits of the parameter type.

In a fifth aspect of the present disclosure, which may be combined with any other aspect listed herein unless specified otherwise, a green color corresponds with acceptable parameter values, yellow areas correspond with parameter values that exceed the soft, upper limits, and red areas correspond with parameter values that exceed the hard, upper limits.

In a sixth aspect of the present disclosure, which may be combined with any other aspect listed herein unless specified otherwise, the text is a green color if it is an acceptable parameter value, the text is a yellow color if it is within the soft limits for the parameter type, and the text is in a red color if it is within the hard limits for the parameter type.

In a seventh aspect of the present disclosure, which may be combined with any other aspect listed herein unless specified otherwise, the memory stores a drug library containing drug entries with upper and lower parameter limits.

In an eight aspect of the present disclosure, which may be combined with any other aspect listed herein unless specified otherwise, a method of operating a PCA pump based on user input includes selecting an infusion therapy type from a preloaded drug library displayed on a display screen, inputting a parameter value, transmitting the infusion therapy type selected and inputted parameter value to a processor, wherein the processor communicates with a memory and determines the lower and upper limits of the parameter type associated with the user-inputted parameter value and user-selected infusion therapy type, transmitting instructions from the memory to the processor for generating a lower and upper limit graphical display on the display screen, comparing the user-inputted parameter value to the instructions received by the processor, generating a graphical display on the display screen displaying the lower and upper limits of the parameter type in different colors, and generating different-colored text for the user-inputted parameter value displayed on the screen based on where the user-inputted parameter value falls within the lower and upper limits of the parameter type

In a ninth aspect of the present disclosure, which may be combined with any other aspect listed herein unless specified otherwise, the preloaded drug library includes information on the upper and lower limits of a parameter type, including the soft limits and hard limits.

In a tenth aspect of the present disclosure, which may be combined with any other aspect listed herein unless specified otherwise, the graphical display is a bar graph.

In an eleventh aspect of the present disclosure, which may be combined with any other aspect listed herein unless specified otherwise, the bar graph has different colors that correspond with acceptable parameter values, soft limit parameter values, and hard limit parameter values.

In a twelfth aspect of the present disclosure, which may be combined with any other aspect listed herein unless specified otherwise, the text is displayed in a green color if it is an acceptable parameter value, the text is displayed in a yellow color if it is within the soft limits for the parameter type, and the text is displayed in a red color if it is within the hard limits for the parameter type.

In a thirteenth aspect of the present disclosure, which may be combined with any other aspect listed herein unless specified otherwise, the processor adjusts the lower and upper limits for a first parameter type based on a user-inputted second parameter type.

In a fourteenth aspect of the present disclosure, which may be combined with any other aspect listed herein unless specified otherwise, a PCA pump includes an actuator, a syringe holder, a display screen a processor, and a memory storing instructions. When the instructions are executed by the processor, the instructions cause the processor to receive a user-selected patient history graph command, retrieve patient history data, retrieve patient history graph instructions, and generate a graph on the display screen that shows the patient history data. The patient data includes infusion therapy intervals (1-, 2-, 4-, 8-, 12-, 24-hour, and cumulative), Loading Dose (LD) & Clinician Bolus (CB) Bolus, PCA Bolus, Programmed Intermittent Epidural Boluses (PIEB), Continuous infusion rate (ml/hr), and Total Volume (ml/hr). In the patient history graph, the infusion therapy intervals (1-, 2-, 4-, 8-, 12-, 24-hour, and cumulative) are displayed on one axis, and the Loading Dose (LD) & Clinician Bolus (CB) Bolus, PCA Bolus, Programmed Intermittent Epidural Boluses (PIEB), Continuous infusion rate (ml/hr), and Total Volume (ml/hr) are displayed on another axis.

In a fifteenth aspect of the present disclosure, which may be combined with any other aspect listed herein unless specified otherwise, the memory stores the patient history data and the patient history data is timestamped.

In a sixteenth aspect of the present disclosure, which may be combined with any other aspect listed herein unless specified otherwise, a method for operating a PCA pump based on user input includes selecting a patient history graph command displayed on a display screen, transmitting the command to a processor, wherein the processor communicates with a memory and retrieves patient history data, transmitting instructions from the memory to the processor for generating a patient history graphical display on the display screen, and generating a graphical display on the display screen displaying the patient history data.

In a seventeenth aspect of the present disclosure, which may be combined with any other aspect listed herein unless specified otherwise, the memory stores patient history data.

In an eighteenth aspect of the present disclosure, which may be combined with any other aspect listed herein unless specified otherwise, the patient history data is timestamped.

In a nineteenth aspect of the present disclosure, which may be combined with any other aspect listed herein unless specified otherwise, the patient history data includes infusion therapy intervals (1-, 2-, 4-, 8-, 12-, 24-hour, and cumulative), Loading Dose (LD) & Clinician Bolus (CB) Bolus, Patient Control Analgesia (PCA) Bolus, Programmed Intermittent Epidural Boluses (PIEB), Continuous infusion rate (ml/hr), and Total Volume (ml/hr).

In a twentieth aspect of the present disclosure, which may be combined with any other aspect listed herein unless specified otherwise, the infusion therapy intervals (1-, 2-, 4-, 8-, 12-, 24-hour, and cumulative) are displayed on one axis of the graphical display, and the Loading Dose (LD) & Clinician Bolus (CB) Bolus, Patient Control Analgesia (PCA) Bolus, Programmed Intermittent Epidural Boluses (PIEB), Continuous infusion rate (ml/hr), and Total Volume (ml/hr) are displayed on another axis of the graphical display.

In a twenty-first aspect of the present disclosure, any of the structure, functionality, and alternatives disclosed in connection with any one or more of FIGS. 1 to 10B may be combined with any other structure, functionality, and alternatives disclosed in connection with any other one or more of FIGS. 1 to 10B.

In light of the present disclosure and the above aspects, it is therefore an advantage of the present disclosure to minimize user interaction with the PCA pump control system to minimize human error.

It is another advantage of the present disclosure to provide a PCA pump user interface that visually aids the user during the programming process to ensure user-inputted parameters are within the lower and upper limits in relation to the infusion therapy type being administered and any interrelation with other parameters inputted by the user.

It is yet another advantage of the present disclosure to provide a PCA pump user interface that generates and displays a real-time patient history graph to aid the user in bedside infusion therapy programming.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a system level diagram of a PCA pump within a hospital information system, according to an example embodiment of the present disclosure.

FIG. 2 is a perspective view of an example PCA pump comprising the Baxter® Novum pump, which may be included within the hospital system of FIG. 1, according to an example embodiment of the present disclosure.

FIG. 3 is a software component diagram of the on-screen guidance operations performed by the PCA pump of FIG. 1, according to an example embodiment of the present disclosure.

FIG. 4 a software component diagram of the patient history operations performed by the PCA pump of FIG. 1, according to an example embodiment of the present disclosure.

FIG. 5 is diagram of a pump control system process for on-screen parameter programming guidance based on user input, according to an example embodiment of the present disclosure.

FIG. 6 is a diagram of a pump control system process to generate a patient history graph based on user input, according to an example embodiment of the present disclosure.

FIG. 7 are sample views of a user interface for on-screen parameter programming guidance for use in the display screen of the PCA pump of FIG. 1, according to an example embodiment of the present disclosure.

FIG. 8 is a sample view of a user interface for on-screen parameter programming guidance for use in the display screen of the PCA pump of FIG. 1, according to an example embodiment of the present disclosure.

FIG. 9 are sample views of a user interface for patient history graphs for use in the display screen of the PCA pump of FIG. 1, according to an example embodiment of the present disclosure.

FIGS. 10A-B are sample detailed views of a user interface for patient history graphs for use in the display screen of the PCA pump of FIG. 1, according to an example embodiment of the present disclosure.

### DETAILED DESCRIPTION

Methods, systems, and apparatus are disclosed herein for a PCA pump user interface that provides on-screen parameter programming guidance and a patient history graph. The example methods, systems, and apparatus are configured to display a color graph and differently-colored text on a PCA pump user interface screen in accordance with the lower and upper limits established for a user-inputted parameter. Additionally, the example methods, systems, and apparatus are configured to generate and display a patient history graph on a PCA pump user interface using real-time patient history data. As such, the user has minimal interaction with the PCA pump control system. For on-screen parameter programming guidance, the user selects the drug being infused and enters a parameter value, and a processor and memory in the PCA pump communicate this information with each other. The processor matches the user-selected drug with an entry in a preloaded drug library. The entry from the preloaded drug library includes information on the upper and lower limits for the parameter inputted by the user. The processor finds the related lower and upper limit graphical instructions, and transmits them to the processor. The processor executes the instructions it receives from the memory, monitoring any changes in the user-inputted parameters, displaying a limits bar graph on the PCA pump screen, and changing the color of the text for the user-inputted parameter values in accordance to where they fall within the lower and upper parameter limits range. As such, there is no need for the user to toggle between screens when inputting a given parameter type, as the PCA pump interface provides visual cues to guide the user in programming.

For patient history graph generation, the user selects a patient history command, and a processor and memory in the PCA pump communicate this information with each other. The processor retrieves patient history and graphical instructions from the memory and transmits them to the processor. The processor executes the instructions it receives from the memory, monitoring any changes in patient history, and generating a patient history graph on the PCA pump screen. As such, there is no need for the user to toggle between screens to understand previously administered treatments when programming a PCA pump bedside, as the patient history graph provides a treatment overview in one screen.

Reference is made herein to a memory. As disclosed herein, a memory refers to a device that holds electronic data and/or instructions for immediate use by a processor and/or pump control system. The memory is able to receive and transmit data.

Reference is made herein to a processor. As disclosed herein, a processor refers to a device that executes instructions stored by the memory. The memory receives and transmits data.

Reference is made herein to an infusion. As disclosed herein, an infusion refers to the putting of fluids, intravenously through the use of a needle or catheter, into the bloodstream. The fluids can be a drug, supplement, or a mix thereof.

Reference is made herein to a drug library. As disclosed herein, a drug library refers to an indexed list of drugs and supplements. Each entry contains the name of the substance (both scientific and branded names), important parameters like maximum and minimum dosages, concentration information, infusion rates, and whether the drug is administered with or without PCA boluses. It should be noted that additional information can be included in the entries for a drug library.

While the example methods, apparatus, and systems are disclosed herein as operating with PCA pumps, it should be appreciated that the methods, apparatus, and systems may be operable with other pumps. For example, the methods, apparatus, and systems may provide for on-screen parameter programming guidance and patient history graph generation in syringe pumps based on user-inputted parameters and commands.

### Medical Environment Embodiment

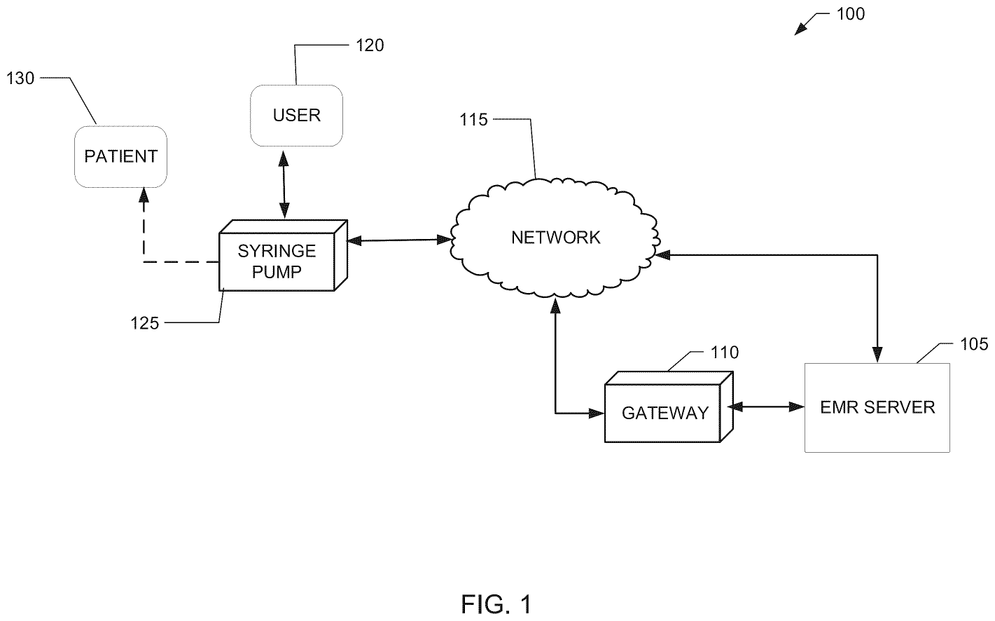

FIG. 1 is a system level diagram of a PCA pump within a hospital information system 100. The example system 100 includes a PCA pump 125, a network 115, a gateway 110, and an Electric Medical Records (“EMR”) server 105. The PCA pump 125 is capable of delivering an intravenous infusion therapy to a patient 130 via one or more intravenous (“IV”) line sets, based on inputs entered by the user 120. The PCA pump 125 connects to the network 115. In addition, the gateway 110 and EMR server 105 also connect to the network 115. As such, the PCA pump 125 is communicatively coupled to the gateway 110 and EMR server 105 via the network 115. In some embodiments, multiple PCA pumps 125 connect to the network 115, the gateway 110, and the EMR server 105. It must be noted that these connections may be wireless, such as via Bluetooth®, or wired, via a serial, Ethernet, CAN, or USB connection.

The gateway 110 is configured to receive infusion therapy type data (e.g., drug name, syringe volume, and bolus volume when applicable) from the PCA pump 125, and route the data to an EMR server 124. In some embodiments, the gateway 110 is configured to convert the data from, for example, EXTCOM message(s) to HL7 message(s). In yet other embodiments, the network 115 and the gateway 110 are omitted from the system 100.

The gateway 110 may also be configured to transmit operating parameters or prescription parameters to the PCA pump 125. For example, the gateway 110 may send an electronic prescription (or software update) to the PCA pump 125 at a predetermined time and/or when the PCA pump 125 is available to accept the prescription. In other instances, the PCA pump 125 may be configured to periodically poll the gateway 110 to determine if an electronic prescription (or software update) is awaiting to be downloaded to the pump.

Relatedly, the PCA pump 125 transmits infusion therapy progress data to the network 110. The network 110 then converts the therapy progress data to a protocol for transmission via Ethernet to the gateway 110 via the network 115. The gateway 110 may include, for example, the Baxter® IQ Enterprise® gateway. As such, the gateway 110 may be configured to integrate with the EMR server 105 or other hospital system to facilitate the transmission of the infusion therapy progress data from the PCA pump 125 to, for example, a hospital electronic medical record (“EMR”) related to the patient 130.

In one embodiment, the EMR server 105 is also communicatively coupled to a pharmacy server (not shown), which is configured to create and/or transmit medication orders corresponding to, for example, a prepared medication (not shown). A medication order includes an electronic record or entry, which identifies a patient (e.g., a patient identifier) and infusion parameters for administration. The medication order is assigned a unique identifier. In some embodiments, the medication order may be printed on a label attached to a medication container that is fluidly coupled to the PCA pump 125. The medication order itself associates a patient identifier with a medication identifier. The EMR server 124 is configured to use the patient identifier in the medication order to store or otherwise associate the medication order with a patient's EMR.

In an alternate embodiment, the system 100 may also include a clinician device (not shown; e.g., a smartphone, tablet computer, laptop computer, workstation, etc.) such that the user 120 could monitor patient data.

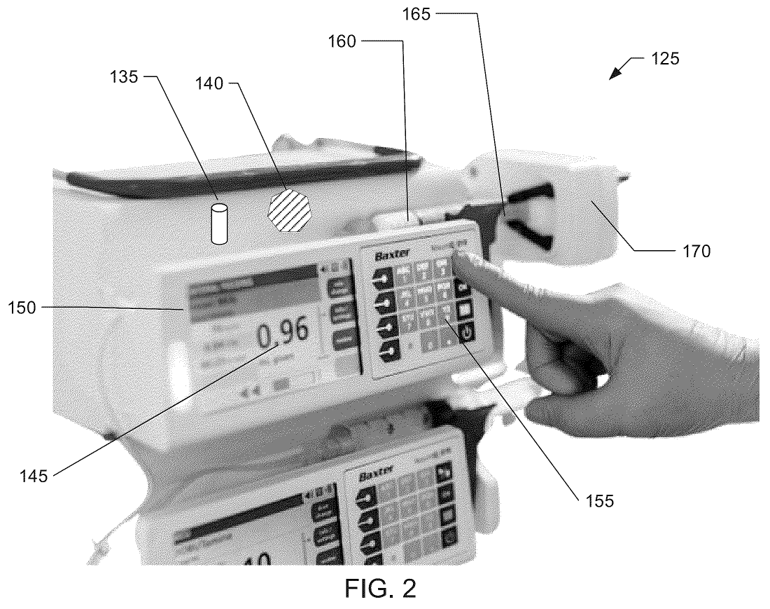

FIG. 2 is a perspective view of an example PCA pump 125. The illustrated infusion pump 125 is the Baxter® Novum IQ PCA pump. In this embodiment, the PCA pump 125 includes a display 150 with interfaces 145 and a keypad 155 to enable a clinician to specify or program an infusion therapy or graphical display command. The PCA pump 125 uses a motor connected to an actuator 170 to actuate a plunger 165 within a syringe 160.

The PCA pump 125 also includes a memory 135 and a processor 140. The memory 135 stores one or more drug libraries, like Dose IQ, that include particular program parameter limits based on care area, dose change, rate of change, drug name, concentration, patient age, patient weight, etc. The limits are configured to ensure that a received prescription or entered infusion therapy is within acceptable ranges and/or limits decided by a medical facility, doctor, or clinician. The drug libraries also include information as to whether an infusion therapy includes PCA boluses, or administers at a continuous constant rate without PCA boluses. The memory 135 also stores patient history data which may include, but is not limited to, a table of different infusion therapy intervals (1-, 2-, 4-, 8-, 12-, 24-hour, and cumulative), Loading Dose (LD) & Clinician Bolus (CB) Bolus, PCA Bolus, Programmed Intermittent Epidural Boluses (PIEB), Continuous infusion rate (ml/hr), and Total Volume (ml/hr). The memory 135 continuously stores real-time patient history data.

The processor 140 is configured to execute machine-readable instructions stored in the memory 135. Execution of the machine-readable instructions by the processor 140 causes the PCA pump 125 to perform the operations described herein.

As noted previously, the PCA pump 125 is connected to and communicates with the gateway 110 (FIG. 1) via the network 115 (FIG. 1). As also noted, the PCA pump 125 is configured to monitor the progress of the infusion therapy and periodically transmit infusion therapy progress data (e.g., medical device data) to the gateway 110 (FIG. 1). The therapy progress data, as disclosed herein, may include, for example, an infusion rate, a dose, a total volume infused, a time remaining for the therapy, a medication concentration, rate change, a volume remaining within a medication container, a medication name, a patient identifier, titration information, bolus information, a care area identifier, a timestamp when the data was generated, an alarm condition, an alert condition, an event, etc. The PCA pump 125 may transmit the data continuously, periodically (e.g., every 30 seconds, 1 minute, etc.), or upon request by the gateway 110 (FIG. 1).

In some embodiments, the PCA pump 125 may also be communicatively coupled to one or more physiological sensors. For example, the PCA pump 125 may be connected to a pulse oximetry sensor, a blood pressure cuff, an access disconnection device, and/or a weight scale. The first pump 125 may be configured to integrate or otherwise include, for example, data from the pulse oximetry sensor into the therapy progress data or, alternatively, transmit the pulse oximetry data separately to the gateway 110 (FIG. 1). The gateway 110 (FIG. 1) can then access the EMR server 105 (FIG. 1) to record this data in a patient's electronic medical record.

### On-Screen Parameter Programming Guidance

As previously noted, at the start of an infusion therapy protocol, the user inputs the infusion therapy type, namely the drug name, into the PCA pump 125. After the user enters the drug name into the PCA pump 125, they must program the various parameters (i.e. the PCA dose amount or the lockout interval).

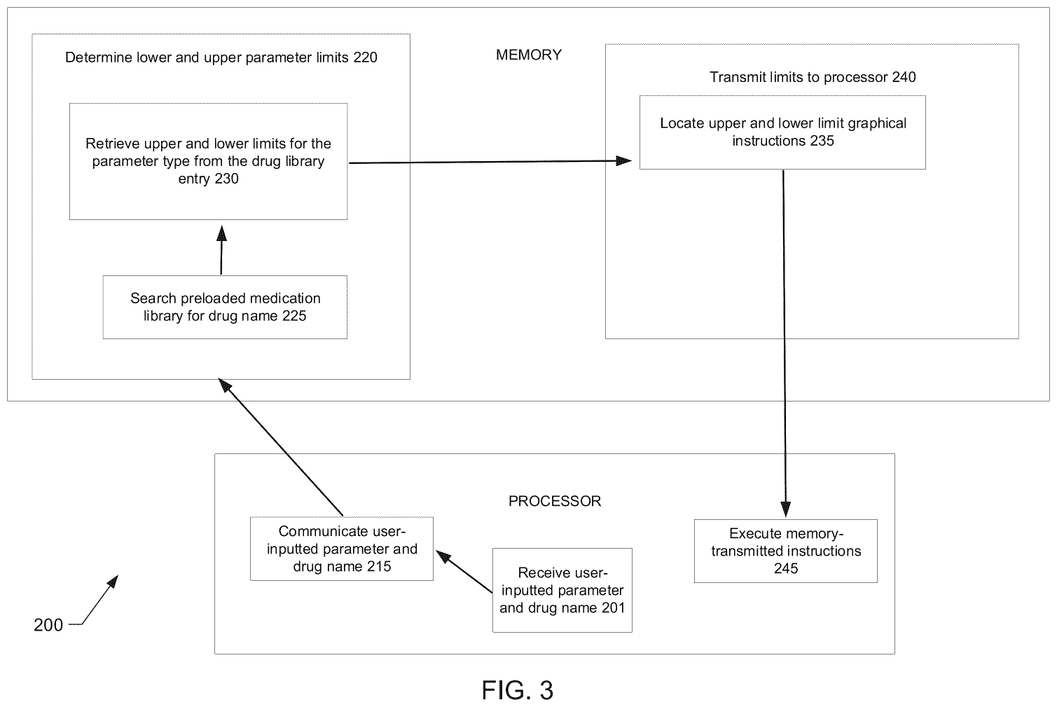

FIG. 3 is a software component diagram of the operations 200 performed by the PCA pump of FIG. 1 to display on-screen parameter programming guidance based on user input in relation to the drug name initially entered by the user. As seen in the diagram in box 201, first the processor 140 receives the user-inputted drug name and parameters (i.e. the PCA dose amount or the lockout interval) data. The parameter is received in the form of value such as a dosage and the drug name is received in the form of a unique identifier. The processor 140 then transmits data containing the user-inputted drug name unique identifier and parameter value 215 to the memory 135. The memory 135 in conjunction with the processor 140 receives unique identifier and searches the drug library for a drug library entry with that matches the unique identifier entered by the user 225. More specifically, the memory 135 in conjunction with the processor 140 indexes its drug library, comparing the drug library entries to the drug or supplement identifier it received.

Upon finding a match, the processor 140 determines the lower and upper limits, including hard and soft limits, of the parameter entered by the user, as it relates to drug name entered by the user 220. More specifically, the processor 140 accesses the matching drug library entry and retrieves data on the upper and lower limits of the parameter entered by the user. The processor 140 then locates upper and lower limit graphical instructions associated with the parameter entered by the user 235, in the memory 135. The memory 135 in conjunction with the processor 140 then transmits the instructions to the processor 240.

The processor 140 receives the memory-transmitted instructions and executes the instructions (block 245). To implement the instructions, the processor compares the parameter value entered by the user to the upper and lower limits it received in the memory-transmitted instructions. The processor does two things concurrently: it generates a colored limits bar graph to display on the user interface, and it also changes the color of the parameter value entered by the user on the interface to indicate whether the parameter value entered is within the range of parameter limits.

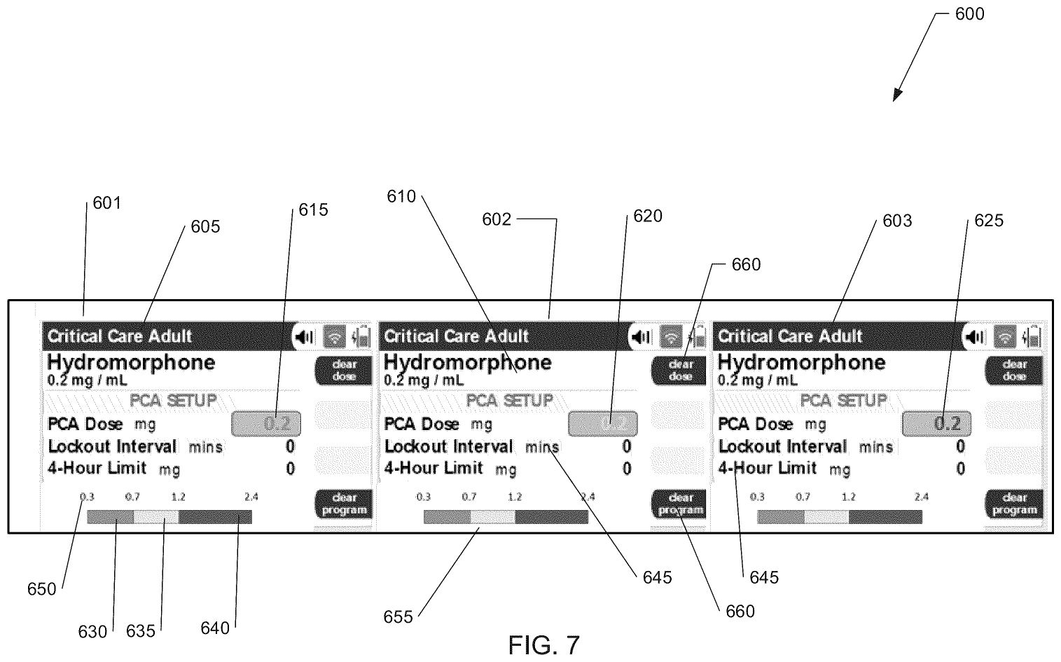

To generate the colored limits bar graph, the processor receives the memory-transmitted instructions and generates a graphic that corresponds with the lower and upper limits for the parameter entered by the user. More specifically, the graphic is, for example, a bar graph horizontally displayed on the bottom of the screen (see FIG. 7) in which there is a green area corresponding with the acceptable ranges of parameter values, a yellow area corresponding with parameter values that exceed the soft, upper limits for the drug, and a red area corresponding with parameter values that exceed the hard, upper limits for the drug.

Concurrently, the processor compares the parameter value entered by the user to the range of limits it received in the memory-transmitted instructions, and displays the parameter value on the screen in green text if the value is within the acceptable ranges. If the parameter value exceeds the soft, upper limits, then the processor displays the value entered in yellow text. If the parameter value exceeds the hard, upper limits, then the processor displays the value entered in red text. It should be noted that the processor continues to compare the parameter value(s) entered by the user as the user changes the parameter value(s). As such, the processor dynamically changes the color of the parameter value text displayed on the screen to show where in the range of values as the user-inputted parameter value changes.

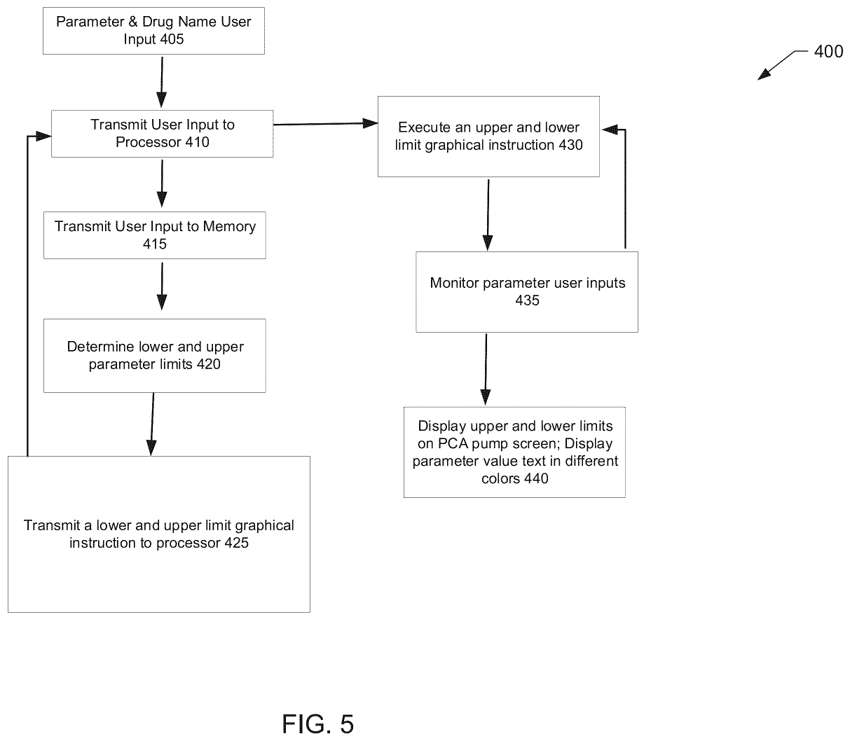

FIG. 5 shows diagram of a pump control system process for on-screen parameter programming guidance based on user input. As seen in the diagram, the user inputs their desired drug name and parameter value, such as the PCA dose amount or the lockout interval (block 405). The user does this by entering, for example, a drug name (an infusion therapy type) and numerical dosage using a keypad and screen display on the PCA pump.

The data corresponding with the user input, a unique drug name and parameter value, is then transmitted to the processor (block 410). Once it arrives at the processor, the processor transmits the user input data to the memory (block 415).

Upon arrival, the memory in conjunction with the processor receives the user input data, locates the unique drug name identifier, and accesses its preloaded drug library to find a matching drug name entry. Upon locating a match, the memory in conjunction with the processor retrieves data associated with the matching entry. The memory in conjunction with the processor then determines the upper and lower limit graphical instructions associated with the parameter value entered by the user (block 420). To achieve this, the memory in conjunction with the processor retrieves from the matching drug name entry, the data indicating lower and upper parameter limits (block 420). More specifically, the lower and upper parameter limits are in the form of soft and hard limits. Soft limits refer to lower or upper limits that are set in the drug library and can be overridden by the user. On the other hand, hard limits refer to lower or upper limits that are set in the drug library and that cannot be overridden by the user.

Upon determining the lower and upper parameter limits the memory in conjunction with the processor then retrieves and transmits lower and upper limit graphical instructions to the processor (block 425).

The processor executes the lower and upper limit graphical instructions after receiving the instructions from the memory (block 430). More specifically, the processor generates a colored limits bar graph which, for example, is a bar graph horizontally displayed on the bottom of the screen (see FIG. 7) with green areas corresponding to the acceptable parameter values, yellow areas corresponding to parameter values that exceed the soft, upper limits, and red areas corresponding to parameter values that exceed the hard, upper limits. At the same time, the processor compares user-inputted parameter value with the lower and upper limits it received from the memory, and displays the user-inputted parameter value in different colors, depending on where it falls within the range of limits. (block 440).

Throughout this time, the processor monitors parameter value(s) entered by the user in two ways (block 435). First, it changes the text color of a given parameter as the user changes the value entered for the given parameter. Additionally, if the user enters a different, new parameter that is interrelated with an initially-entered parameter value, the processor determines how, if at all, the newly-entered parameter affects the lower and upper limits of the initially-entered parameter and changes the text color of the parameter values accordingly.

FIG. 7 shows diagrams of a user interface for use in the display screen of the PCA pump of FIG. 1. As seen, the user interface can display a user-inputted parameter value in a green text 601, a yellow text 602, or a red text 603.

As seen in FIG. 7, on-screen parameter programming guidance user interface shows the drug name of the infusion therapy 610, the care area 605, and various command control buttons 660, like dosage or program clearing. The interface also shows the various parameters 645 that the user can input when programming the PCA pump.

Notably, the user interface shows a bar graph 655, which corresponds with the lower and upper limits for a given parameter. As seen, the bar graph 655 includes a green section 630, a yellow section 635, and a red section 640. Above each section, there are numbers 650, which denote the values at which a parameter's range changes from one color to another. More importantly, as noted above, the green section corresponds with the acceptable parameter values, the yellow section corresponds with values that exceed the soft, upper limits for the parameter, and the red section corresponds with values that exceed the hard, upper limits for the parameter.

Relatedly, as explained above, the user interface displays the user-inputted parameter values in different colors based on where the value falls within the lower and upper limits. For example, in one screen 601, the user-inputted parameter 615 is displayed in green text because it falls within an acceptable value in the lower and upper limits. However, in another screen 602, the user in-putted parameter 620 is displayed in yellow because it exceeds the soft limits for the given parameter. Finally, in another screen 603, user in-putted parameter 625 is displayed in red because it exceeds the hard limits for the given parameter. In turn, as evident from the various text colors 601, 602, 603, the user interface provides visual cues to signal to the user whether their parameter inputs are within the safe lower and upper limits. As an additional visual aid, the user interface displays a bar graph 655 that also displays the acceptable lower and upper limits for a given parameters with colors and accompanying numbers. It should be noted that the colors and/or visual cues used may vary in different embodiments.

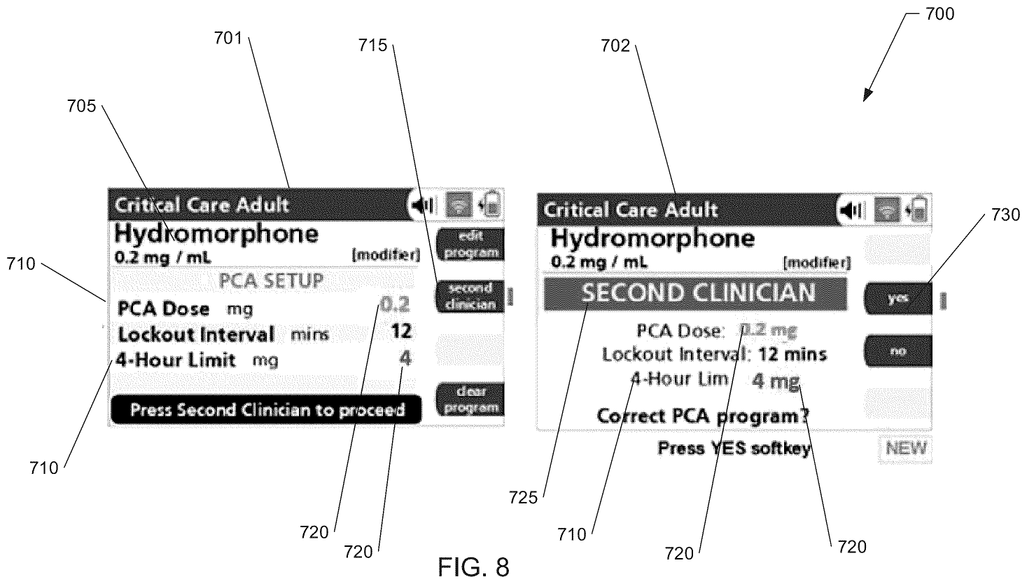

In this embodiment, the on-screen parameter programming guidance user interface also includes a screen in which a second user can visually verify the programming parameters inputted by a first user. FIG. 8 shows diagrams of the user verification screens. The first screen 701 shows an overview of the programmed parameters as entered by the first user. More specifically, the screen shows the drug name 705, the parameter types 710, and the parameter values entered by the first user 720. As seen, the parameter values 720 are displayed in colors in the same manner as described for FIG. 7 above. The first screen also includes a command button 715 to allow the second user to denote that they are reviewing the first user's programming.

The second screen 702 includes the same information as the first screen 701. However, the second screen includes a colorful banner 725 that denotes a second user is reviewing previously-programmed parameters. In addition, the second screen 702 includes command buttons that allow the second user to correct/change the programming parameters entered by the first user.

As such, the on-screen parameter programming guidance on this user interface dynamically shows not only whether a given user-inputted parameter value is within the lower and upper limits for the drug being infused into the patient based on text color, but also accounts for the interrelation of multiple programming parameters. In turn, the user is able to program the PCA pump with the help of the user interface color cues, and without having to toggle between screens.

### Patient History Graph

As previously noted, throughout an infusion therapy protocol, the user monitors and adjusts programming parameters on the PCA pump 125. The programming adjustments the users make depend on the user accessing and interpreting patient history on the PCA pump.

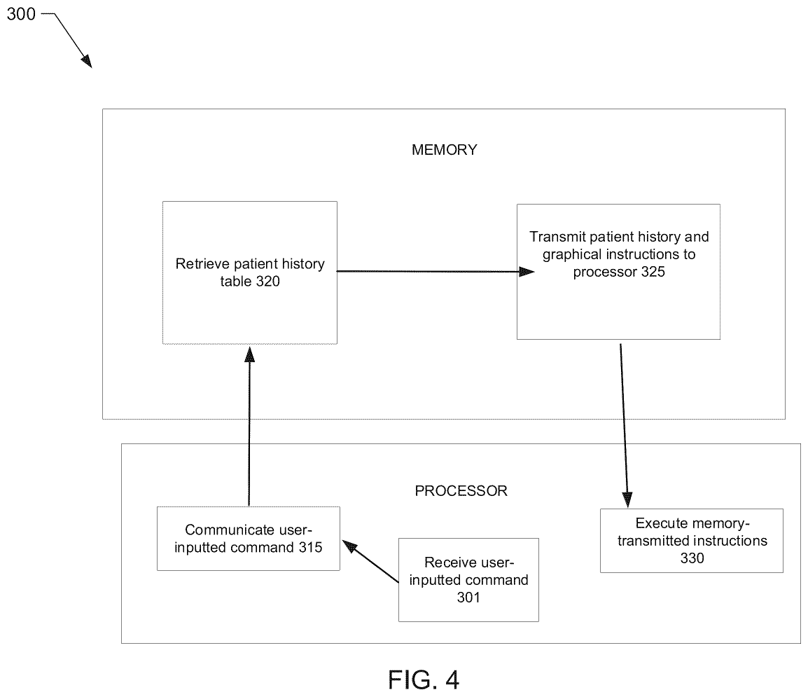

FIG. 4 is a software component diagram of the operations 300 performed by the PCA pump of FIG. 1 to display patient history based on user-inputted commands. As seen in the diagram, first the processor 140 receives the user-inputted command to display a patient history graph 301. The user, for example, inputs the command by using the keypad on the PCA pump to select a “patient history”, or the like, option on the screen. The processor 140 then transmits the command to the memory 315 in the form of a command to access patient data stored in the memory during the infusion therapy protocol. It should be noted that patient history data is continually being collected and stored in the memory by the PCA control system. As previously noted, the patient history data may include, but is not limited to, information at different infusion therapy intervals (1-, 2-, 4-, 8-, 12-, 24-hour, and cumulative), such as Loading Dose (LD) & Clinician Bolus (CB) Bolus, PCA Bolus, Programmed Intermittent Epidural Boluses (PIEB), Continuous infusion rate (ml/hr), and Total Volume (ml/hr).

At this point, the memory 135 in conjunction with the processor 140 receives the command, searches for the patient history, retrieves the patient history, and compiles the patient history in a table 320. Then, the memory in conjunction with the processor transmits the patient history and graph-generation instructions to the processor 325.

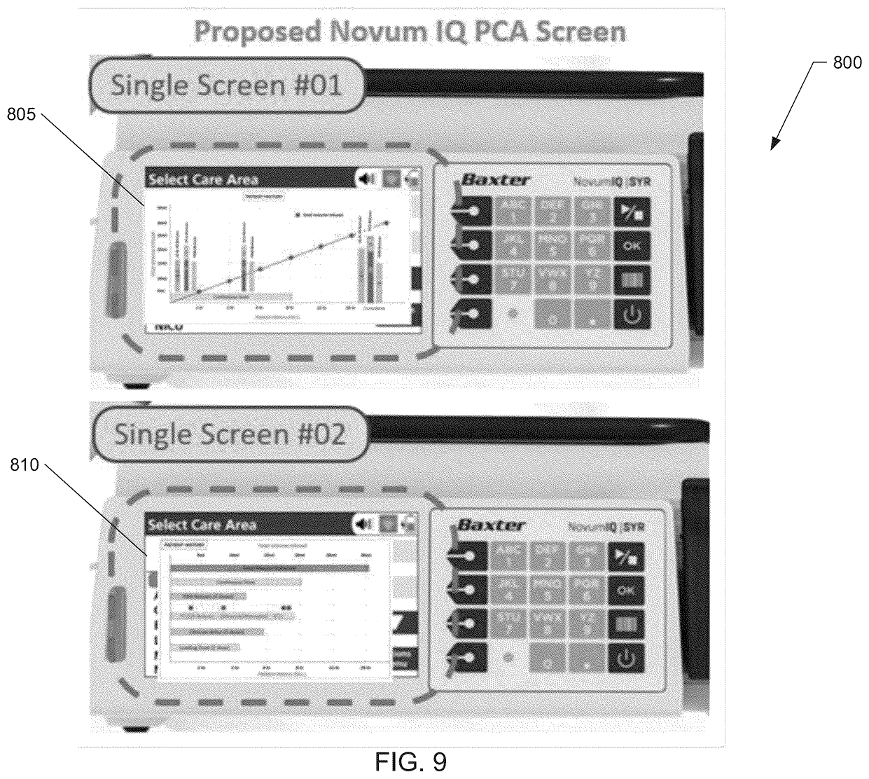

The processor 140 receives the memory-transmitted instructions and patient history data, and executes them. To implement the instructions, the processor generates a graphical color display of the patient history data, which is then transmitted and displayed on the PCA pump screen. This includes, for example, calculating and displaying trend lines based on the patient history the processor receives. More specifically, the graphic is, for example, a bar graph using various colors to denote different parameters and/or intervals (see FIG. 9). Notably, the user can modify the specific patient history parameters displayed on the resulting graph. Additionally, the user can change the type of graph (i.e. horizontal or vertical) displayed on the PCA pump screen.

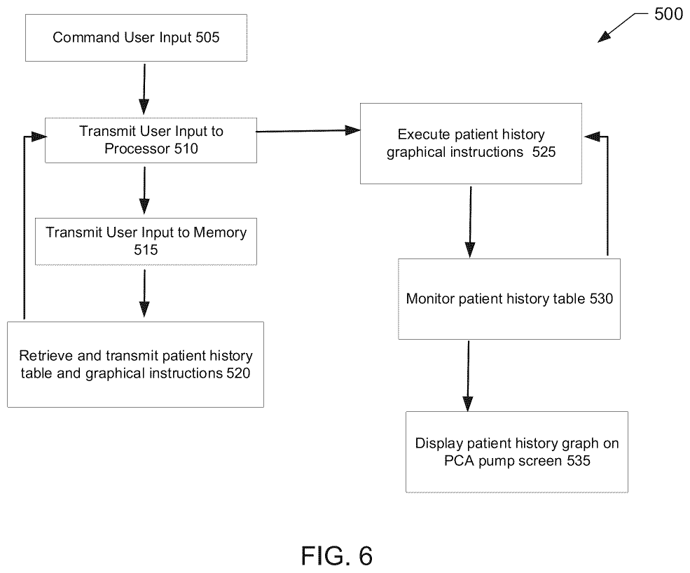

FIG. 6 shows diagram of a pump control system process for displaying patient history data based on user input. As seen in the diagram, the user inputs a command (block 505). The user does this by selecting, for example, a “Patient History” option on the keypad and screen display on the PCA pump.

The data corresponding with the user input, a command, is then transmitted to the processor (block 510). Once it arrives at the processor, the processor transmits the command to the memory (block 515).

Upon arrival, the memory in conjunction with the processor receives the command, locates the patient history, retrieves the patient history, and compiles the patient history in a table (block 520). Concurrently, the memory in conjunction with the processor retrieves graph-generating instructions and transmits them to the processor (block 520).

After receiving the patient history table and graph-generating instructions, the processor executes the graph-generating instructions (block 520). More specifically, the processor generates a graphical color display using the data it received in the patient history table. The processor generates the graphical color display and then transmits and displays it on the PCA pump screen. While generating the graphical color display, the processor performs various dynamic operations. For example, the processor uses the patient history table to calculate trend line, which could include using data points to run linear regressions for display on the PCA pump screen. Additionally, the processor continuously monitors the memory for any new, incoming patient history table data and generates an updated graphical color display according any new data. The processor also generates different graphical color displays based on user input, which may include changing the orientation of the graph and/or changing the patient history parameters on the graph (see FIG. 9).

FIG. 9 shows diagrams of a patient history graph in the display screen of the PCA pump of FIG. 1. As seen, the patient history graph can be displayed in multiple configurations 805, 810.

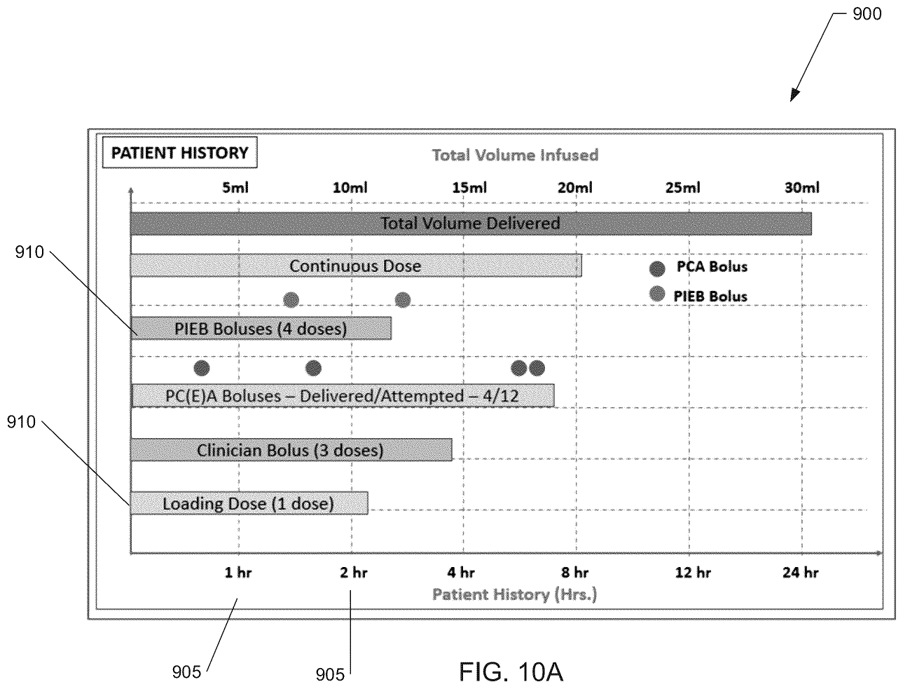

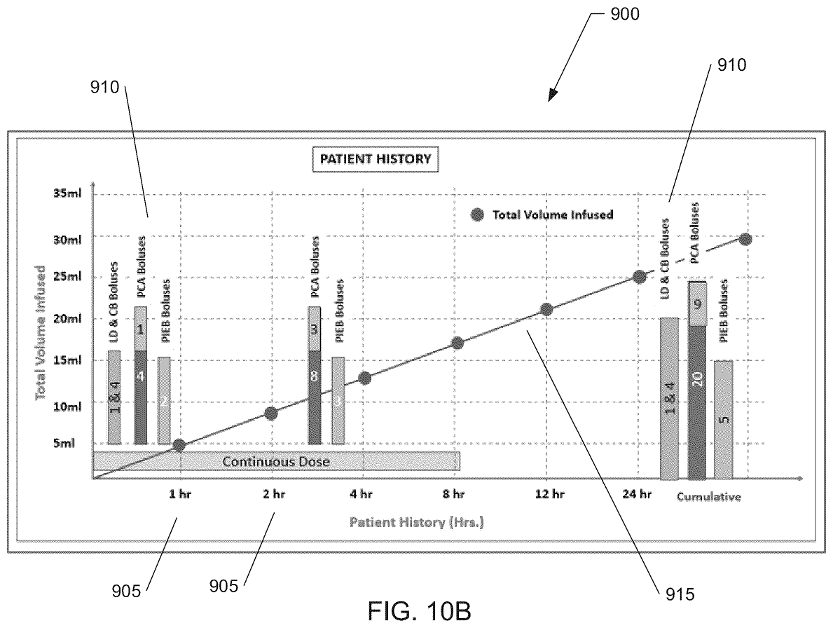

FIGS. 10A-B show more detailed views of a patient history graph in the display screen of the PCA pump of FIG. 1. As seen in FIG. 10A, the patient history graph 900, can include the patient history over various time intervals 905, in addition to various other parameters 910.

As a contrast, in FIG. 10B, the patient history graph 900 can include overlaid information such as trend lines 915, on top of the various time intervals 905 and other parameters 10. Additionally, as seen in FIGS. 10A and 10B, the orientation of the patient history graph can vary. It should be noted that the colors, line types, orientations, and other visual elements can vary from those shown in FIGS. 10A and 10B.

As such, the patient history graphical display on this user interface shows a complete overview of a given patient's infusion history, with the user being able to modify the patient history parameters displayed on the PCA pump. In turn, the user is able to quickly assess a patient's treatment and make any modifications at the patient's bedside without having to toggle between screens, thereby reducing the chances of programming errors that could cause patient discomfort.

### CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A PCA pump comprising:
an actuator;
a syringe holder;
a display screen;
a processor; and
a memory storing instructions, which when executed by the processor, cause the processor to
receive a user-selected infusion therapy type and a user-inputted parameter value,
receive the lower and upper limits of the parameter type associated with the user-inputted parameter value and user-selected infusion therapy type,
compare the user-inputted parameter value to the lower and upper limits of the parameter type,
generate a graph on the display screen that shows the lower and upper limits of the parameter type in different colors, and
generate different-colored text for the user-inputted parameter value based on where the user-inputted parameter value falls within the lower and upper limits of the parameter type.

2. The PCA pump of claim 1, wherein the upper and lower limits of the parameter type include the soft limits and hard limits.

3. The PCA pump of claim 2, wherein the graph is a bar graph.

4. The PCA pump of claim 3, wherein the different colors in the bar graph correspond with the lower and upper limits of the parameter type.

5. The PCA pump of claim 4, wherein a green color corresponds with acceptable parameter values, yellow areas correspond with parameter values that exceed the soft, upper limits, and red areas correspond with parameter values that exceed the hard, upper limits.

6. The PCA pump of claim 2, wherein the text is a green color if it is an acceptable parameter value, the text is a yellow color if it is within the soft limits for the parameter type, and the text is in a red color if it is within the hard limits for the parameter type.

7. The pump of claim 1, wherein the memory stores a drug library containing drug entries with upper and lower parameter limits.

8. A method for operating a PCA pump based on user input comprising:
selecting an infusion therapy type from a preloaded drug library displayed on a display screen;
inputting a parameter value;
transmitting the infusion therapy type selected and inputted parameter value to a processor, wherein the processor communicates with a memory and determines the lower and upper limits of the parameter type associated with the user-inputted parameter value and user-selected infusion therapy type;
transmitting instructions from the memory to the processor for generating a lower and upper limit graphical display on the display screen;
comparing the user-inputted parameter value to the instructions received by the processor;
generating a graphical display on the display screen displaying the lower and upper limits of the parameter type in different colors; and
generating different-colored text for the user-inputted parameter value displayed on the screen based on where the user-inputted parameter value falls within the lower and upper limits of the parameter type.

9. The method of claim 8, wherein the preloaded drug library includes information on the upper and lower limits of a parameter type, including the soft limits and hard limits.

10. The method of claim 8, wherein the graphical display is a bar graph.

11. The method of claim 10, wherein the bar graph has different colors that correspond with acceptable parameter values, soft limit parameter values, and hard limit parameter values.

12. The method of claim 8, wherein the text is displayed in a green color if it is an acceptable parameter value, the text is displayed in a yellow color if it is within the soft limits for the parameter type, and the text is displayed in a red color if it is within the hard limits for the parameter type.

13. The method of claim 8, wherein the processor adjusts the lower and upper limits for a first parameter type based on a user-inputted second parameter type.

14. A PCA pump comprising:
an actuator;
a syringe holder;
a display screen;
a processor; and
a memory storing instructions, which when executed by the processor, cause the processor to
receive a user-selected patient history graph command,
retrieve patient history data,
retrieve patient history graph instructions, and
generate a graph on the display screen that shows the patient history data,
wherein the patient data includes infusion therapy intervals (1-, 2-, 4-, 8-, 12-, 24-hour, and cumulative), Loading Dose (LD) & Clinician Bolus (CB) Bolus, Patient Control Analgesia (PCA) Bolus, Programmed Intermittent Epidural Boluses (PIEB), Continuous infusion rate (ml/hr), and Total Volume (ml/hr), and
the infusion therapy intervals (1-, 2-, 4-, 8-, 12-, 24-hour, and cumulative) are displayed on one axis, and the Loading Dose (LD) & Clinician Bolus (CB) Bolus, Patient Control Analgesia (PCA) Bolus, Programmed Intermittent Epidural Boluses (PIEB), Continuous infusion rate (ml/hr), and Total Volume (ml/hr) are displayed on another axis.

15. The PCA pump of claim 14, wherein the memory stores the patient history data and the patient history data is timestamped.

16. A method for operating a PCA pump based on user input comprising:
selecting a patient history graph command displayed on a display screen;
transmitting the command to a processor, wherein the processor communicates with a memory and retrieves patient history data;
transmitting instructions from the memory to the processor for generating a patient history graphical display on the display screen; and
generating a graphical display on the display screen displaying the patient history data.

17. The method of claim 16, wherein the memory stores patient history data.

18. The method of claim 17, wherein the patient history data is timestamped.

19. The method of claim 16, wherein the patient history data includes infusion therapy intervals (1-, 2-, 4-, 8-, 12-, 24-hour, and cumulative), Loading Dose (LD) & Clinician Bolus (CB) Bolus, Patient Control Analgesia (PCA) Bolus, Programmed Intermittent Epidural Boluses (PIEB), Continuous infusion rate (ml/hr), and Total Volume (ml/hr).

20. The method of claim 19, wherein the infusion therapy intervals (1-, 2-, 4-, 8-, 12-, 24-hour, and cumulative) are displayed on one axis of the graphical display, and the Loading Dose (LD) & Clinician Bolus (CB) Bolus, Patient Control Analgesia (PCA) Bolus, Programmed Intermittent Epidural Boluses (PIEB), Continuous infusion rate (ml/hr), and Total Volume (ml/hr) are displayed on another axis of the graphical display.
